(19) United States
(12) Patent Application Publication
(10) Pub. No.: US 2020/0194130 A1
(43) Pub. Date: Jun. 18, 2020

(54) METHOD AND DEVICE FOR ESTIMATING A LOCAL PARTICLE CONCENTRATION

(71) Applicant: KONINKLIJKE PHILIPS N.V., EINDHOVEN (NL)

(72) Inventors: Michael Martin SCHEJA, SHANGHAI (CN); Declan Patrick KELLY, SHANGHAI (CN); Cornelis Reinder RONDA, AACHEN (DE)

(21) Appl. No.: 16/473,269

(22) PCT Filed: Dec. 27, 2017
(86) PCT No.: PCT/EP2017/084596

§ 371 Date: Jun. 25, 2019

(30) Foreign Application Priority Data
Dec. 27, 2016 (CN) PCT/CN2016/000713
Jan. 24, 2017 (EP) 17152736.9

(51) Int. Cl. G16H 50/80; G06F 30/25

(52) U.S. Cl. CPC G16H 50/80; G06F 30/25

(57) ABSTRACT

The present invention relates to a device and method for estimating a local particle concentration indicating the local concentration of pollen and/or microorganisms and to a device and method for generating or refining a particle concentration map of a region. To increase the resolution and accuracy and to enable tracking and monitoring of exposure of a user a particle concentration map (40, 40′) of a region (30) including the actual location (33) is used, which may be generated and refined by crowdsourcing.

### FIELD OF THE INVENTION

The present invention relates to a method and a device for estimating a local particle concentration at an actual location indicating the local concentration of pollen and/or microorganisms. Further, the present invention relates to a method and a device for generating or refining a particle concentration map of a region indicating the concentration of pollen and/or microorganisms.

### BACKGROUND OF THE INVENTION

Pollen represent a significant trigger for allergies. They may also worsen chronic respiratory diseases, such as asthma. Therefore, there is a strong need to quantify pollen concentrations and make those data available for affected groups. Some websites such as pollen.com provide this service by publishing pollen data which they receive from professional monitoring stations (also called “particle count locations” herein). Those pollen counts are usually obtained by collecting pollen from air (e.g. over 24 hours) and then counting and analyzing the samples under a microscope. Since pollen concentration in air is usually quite low (a concentration of 20 grains/m³ is already considered as “high” for grass pollen according to the NAB scale as shown in table 1), monitoring stations use professional equipment to pre-concentrate the pollen by immobilizing them on a substrate.

Where such atmospheric pollen counts are reported, a single monitoring station ordinarily serves an entire city or region. Hence, the spatial coverage of those stations is not very high. Furthermore, pollen concentrations in the air can vary significantly based on the location. Therefore, these published data give only a rough indication about the daily pollen level of a bigger region, but do not represent the actual exposure of a specific subject. Personal exposure tracking as disclosed would overcome this problem. However, pollen sensors hardly exist, especially for home use, and existing pollen sensors have a complex device architecture and a relatively large size.

Similar thoughts are valid for the detection and monitoring of microorganisms present e.g. in water, e.g. to determine the microbiological purity of water, whether the microorganisms present in the water investigated are potentially harmful or to investigate the microbiological purity of air, liquids and surfaces in e.g. hospitals.

### SUMMARY OF THE INVENTION

It is an object of the present invention to provide a method and a device for estimating a local particle concentration indicating the local concentration of pollen and/or microorganisms with high spatial resolution and accuracy and at low costs.

It is an object of the present invention to provide a method and a device for generating or refining a particle concentration map of a region indicating the concentration of pollen and/or microorganisms with high spatial resolution and accuracy and at low costs. The method may be a computer implemented method whereby different steps of the method are executed by a processing unit.

In a first aspect of the present invention a method of estimating a local particle concentration at an actual location is presented comprising

receiving particle count information, e.g. digital particle count information or data, indicating a recent particle count at one or more particle count locations,

receiving or generating a particle concentration map of a region, e.g. a digital particle concentration map of a region, including the actual location, said particle concentration map including relative particle concentration information indicating, per sub-region of a number of sub-regions of the region, the particle concentration at the sub-region relative to a particle count at one or more particle count locations,

determining the sub-region, in which the actual location is located, and

determining the local particle concentration at the actual location based on the relative particle concentration information of the determined sub-region and the received particle count information.

According to an embodiment of the invention, the method may comprise a step of receiving or generating location data related to the actual location, e.g. location of a user running the method on his/her smartphone.

The particle count information may be received wired or wirelessly. For example, the particle count information is received by a wireless or wired data transfer component or chip. The particle count information is thereafter transferred to a processor. The particle count information may come from particle counting stations such as pollen sensing stations positioned at different locations. The information may also come from a plurality of users, e.g. crowdsourced data. The particle count information comprises information on the amount of particles in one or more locations. Thereafter, the processor processes the particle count information and generates a particle concentration map of a region. This particle concentration map contains particle count information of different sub-regions of the region. Each sub-region covers a certain area of the region. All sub-regions cover the complete region. A particle count at a certain sub-region is defined as a difference with another sub-region. Thus, the particle concentration map includes relative particle concentration information indicating, per sub-region the particle concentration at the sub-region relative to a particle count at one or more particle count locations. Further, the actual location may be determined within the method itself, for example by determining the actual location via GPS based techniques or via IP address location techniques. When the actual location is determined or received, the sub-region related to the actual location is identified using the processor. When the sub-region is identified, the processor determines the particle concentration at the actual location using the relative particle concentration information of the identified sub-region and the received particle count information.

In a further aspect of the present invention a method of generating or refining a particle concentration map of a region is presented comprising

measuring and/or receiving local particle count information, e.g. digital local particle count information or data, indicating the particle count at the actual location,

receiving particle count information, e.g. digital particle count information, indicating a recent particle count at one or more particle count locations, and

generating or refining a particle concentration map of a region, e.g. a digital particle concentration map of a region, including the actual location, said particle concentration map including relative particle concentration information indicating, per sub-region of a number of sub-regions of the region, the particle concentration at the sub-region relative to a particle count at one or more particle count locations, wherein the particle concentration map is generated or refined based on the local particle count information and the received particle count information.

The method of generating or refining a particle concentration map of a region may be a computer implemented method whereby the different steps are performed by one or more processors of a device. For example, the method may be software such as an app running on a smartphone. The different steps of the method may be implemented by a processor of the device on which the software is running

Measuring local particle count information may be performed by a particle counter/sensor. Such a counter/sensor may be part of a device implementing the method. The information receiving may be done using a data transfer component/chip which may be part of the device implementing the method. Receiving particle count information indicating a recent particle count at one or more particle count locations may be performed by the data transfer component/chip of a device implementing the method. Generating or refining a particle concentration map of a region including the actual location may be performed by a processor of a device implementing the method. The method may comprise a step of receiving or generating data related to the actual location using e.g. a GPS chip or IP address location searching techniques.

In yet further aspects of the present invention, there are provided corresponding devices, a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the all the claimed methods, device, computer program and medium have similar and/or identical preferred embodiments as defined in the dependent claims of the claimed method and as disclosed herein.

The present invention is based on the idea to provide a digital solution using crowdsourcing-based pollen and/or microorganism measurements and the location data (e.g. from GPS) acquired during sampling to realize a number of additional benefits such as further increase in spatial resolution, accuracy of the data, and indication of precision, without involving much costs for additional hard- and/or software. Once available, such data can be leveraged to improve existing and future devices, method or computer programs (such as an “app”) for asthma and allergy management, e.g. by providing user's valuable information about actual exposure and how to reduce/avoid exposure.

Based on particle count information from one or more locations, a particle concentration map of a region is generated which includes relative particle concentration information per sub-region of the region whereby the particle concentration at the sub-region is relative to a particle count at one or more particle count locations. The advantage of such a particle concentration map containing relative particle count information is that when particle count information from one sub-region is received, particle count information from other sub-regions is updated automatically without having to retrieve particle count information from those other sub-regions. The relative particle count information is used to estimate the particle count at a certain sub-region. This technique allows a fast updating of the particle count information of different sub-regions in the particle concentration map without having to process or retrieve large amounts of particle count information data of all those sub-regions. This increases the accuracy of the data presented to the user. It also reduces power consumption of a device implementing the method as less particle count data must be retrieved and as less particle count data must be processed while still being able to provide an updated particle concentration map. Further, to the user it provides a very efficient and fast manner of providing a particle concentration map of a region that is updated whenever particle count information from one or more locations is retrieved.

Generally, the local particle concentration at the actual location is based on the relative particle concentration information of the determined sub-region, in which the actual location is located, and the received particle count information indicating a recent particle count (also called “benchmark count”) at one or more particle count locations (e.g. professional monitoring stations, also called “benchmark locations”). In one embodiment the local particle concentration at the actual location is determined based on the relative particle concentration information of the determined sub-region and the received particle count information at the particle count location closest to the actual location, i.e. only a single particle count information is used, namely the one from the particle count location closest to the actual location.

The local particle concentration at the actual location may be determined by extrapolating the particle count indicated by the received particle count information at the particle count location closest to the actual location based on the relative particle concentration information of the determined sub-region, which provides a simple way of determining the local particle concentration at the actual location.

In another embodiment the local particle concentration at the actual location is determined based on the relative particle concentration information of the determined sub-region and the received particle count information at two or more particle count locations closest to the actual location. For instance, per particle count location a preliminary local particle concentration may be formed, from which an average is computed representing the final local particle concentration.

Hereby, in a simple manner, the local particle concentration at the actual location may be determined by extrapolating, individually per particle count location, the particle count indicated by the received particle count information based on the respective relative particle concentration information of the determined sub-region with respect to the respective particle count location and by averaging the extrapolated particle counts, in particular by averaging or weighted averaging the extrapolated particle counts.

The particle concentration map may include relative particle concentration information indicating, per sub-region of a number of sub-regions of the region, a deviation of the particle concentration at the sub-region from the particle count at one or more particle count locations in absolute or relative terms. Thus, as long as the local position of the user belongs to any sub-region of the complete region covered by the particle concentration map the local particle concentration can be easily determined if at least one particle count from one or more particle count locations is known.

Since the particular concentration changes over time, e.g. the amount of pollen in the air changes with the seasons of the year and in some seasons even every day, in an embodiment the particle concentration map may include relative particle concentration information for different times, in particular different weeks and/or days and/or hours, over the year.

In another embodiment the particle concentration map may include relative particle concentration information for different kinds of particles, e.g. different kinds of pollen, so that a user can select the desired kind of particle, for which the local particle concentration shall be determined or local particle concentrations for various kinds can be determined.

The method may further comprise the step of receiving particle calendar information indicating typical particle concentrations at various regions per time, wherein the local particle concentration at the actual location is determined based on the relative particle concentration information of the determined sub-region, the received particle count information and the received particle calendar information at the determined sub-region. This further improves the accuracy of the local particle concentration.

In still another embodiment the method may further comprise the step of measuring or receiving local particle count information indicating the particle count at an actual location, wherein the particle concentration map is generated or refined based on the local particle count information and the received particle count information indicating a recent particle count at one or more particle count locations. Thus, the user of the method may even further improve or update the particle concentration map, which may even be shared with other users.

According to another aspect of the present invention a method of generating or refining a particle concentration map of a region indicating the concentration of pollen and/or microorganisms is presented. Said method uses local particle count information indicating the particle count at the actual location and particle count information indicating a recent particle count at one or more particle count locations to generate or refine a particle concentration map. This method may be further improved, in particular in respect of accuracy and resolution of the particle concentration map, in an embodiment, in which further local particle count information indicating the pollen count at one or more further locations is measured and/or received, wherein further local particle count information include location information indicating the location of measurement of the respective local particle count information, and in which the further local particle count information is used in the generation and/or refinement of the particle concentration map.

The present invention may be implemented in hard- and/or software, e.g. in form of an application program for an electronic user device, such as a PC, laptop, tablet, smartphone, smart watch, etc.

According to another aspect, the invention relates to a method and device for estimating a particle concentration at a position, comprising:

receiving particle concentration information from a plurality of locations;

generating a particle concentration map of a region using the received particle concentration information;

receiving a position;

determining particle concentration at the received position from the particle concentration map;

characterized by receiving benchmark particle concentration information from a benchmark location,
wherein the particle concentration map is generated using the particle concentration information relative to the benchmark particle concentration information,
wherein the particle concentration map of the region is updated when new benchmark particle count information is received and
wherein the particle concentration at the received position is determined using a most recent updated particle concentration map.

Hereby, the particle concentration map may be generated by translating the particle concentration map into a relative value map by normalizing the value at each location to the value at the benchmark location, so that the particle concentration at each location is expressed as a percentage of the concentration at the benchmark location.

Further, the particle concentration map of the region may be updated by receiving benchmark particle concentration information from a benchmark location and creating an updated concentration map by deriving the concentration value at each target location from the corresponding percentage and the updated benchmark value.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of a device for estimating a local particle concentration according to the present invention,

FIG. 2 shows a diagram illustrating a region indicating the locations of a user and of several particle count locations,

FIG. 3 shows a first embodiment of a particle concentration map according to the present invention,

FIG. 4 shows the map of the region filled with actual values based on the particle concentration map shown in FIG. 3,

FIG. 5 shows a second embodiment of a particle concentration map according to the present invention,

FIG. 6 shows a schematic diagram of a device for generating or refining a particle concentration map of a region according to the present invention, and

FIG. 7 shows a schematic diagram of a system including the various devices carrying out aspects of the invention.

### DETAILED DESCRIPTION OF THE INVENTION

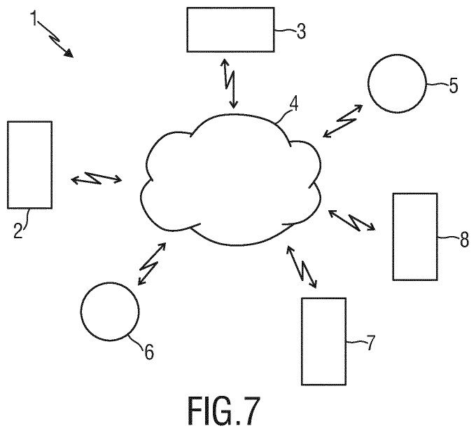

Before details of various embodiments of the present invention are described, the general layout of an exemplary system including the various devices carrying out aspects of the invention shall be explained by way of FIG. 7. The system 1 comprises a user device 2, such as a smartphone, tablet, smart watch or a dedicated device for estimating the local particle concentration at the actual location of the user device 2. The system 1 further comprises a remote device 3, such as a server, which is accessible by the user device 2 via a network 4, e.g. a computer network, the internet, a communications network, etc. The system 1 further comprises one or more particle count locations 5, 6 (also called “benchmark locations”), such as professional particle count measuring stations. Further, a number of further user devices 7, 8 of further users may be part of the system 1.

The particle concentration at the actual location of the user device 2 may be estimated within the remote device 3 or within the user device 2 or commonly in both devices, wherein both devices 2 and 3 perform a respective part of the steps required for the estimation. The particle concentration map used in the estimation of the particle concentration at the actual location of the user device 2 may also be generated within the remote device 3 or within the user device 2 or commonly in both devices, wherein both devices 2 and 3 perform a respective part of the steps required for the estimation. In a likely scenario, the particle concentration map is generated and updated within the remote device 3 and also the particle concentration at the actual location of the user device 2 is estimated within the remote device 3.

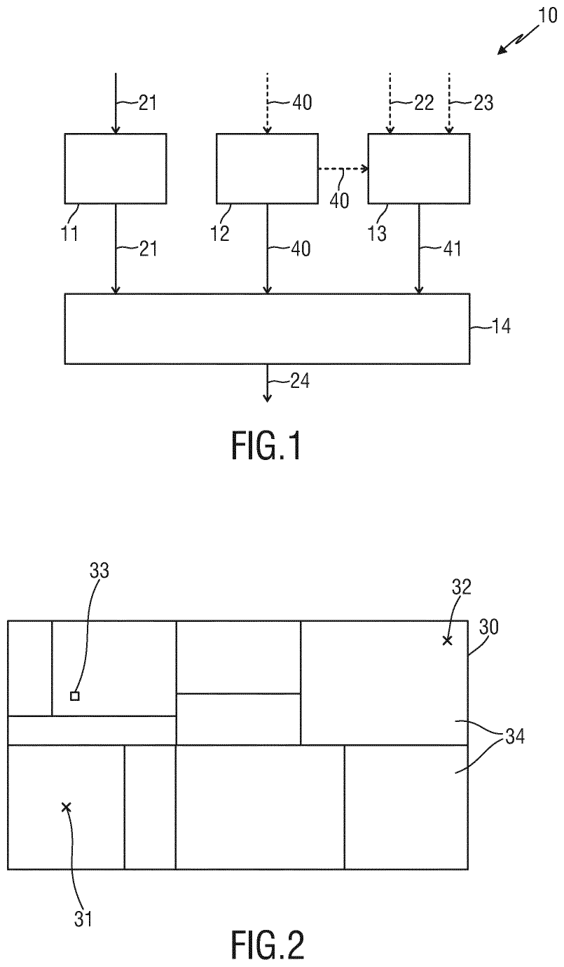

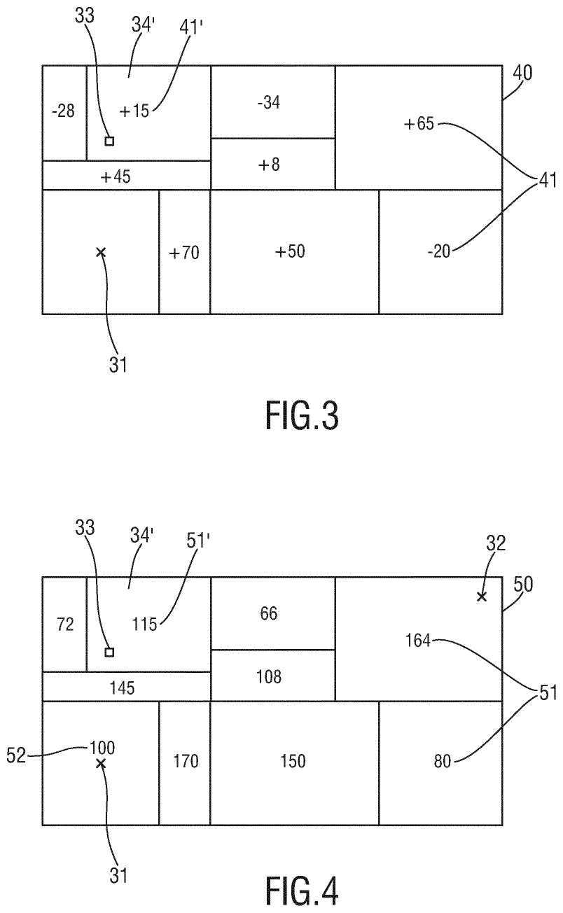

By way of FIGS. 1 to 3 a first aspect of the present invention will be explained. FIG. 1 shows a schematic diagram of a device 10 for estimating a local particle concentration according to the present invention, which represents an embodiment of the user device 2 in this example. FIG. 2 shows a diagram illustrating a region 30 indicating the locations of a user and of several particle count locations. FIG. 3 shows a first example of a particle concentration map 40 according to the present invention.

The device 10 comprises a particle count information input 11, e.g. a wireless data interface, for receiving particle count information 21 indicating a recent particle count at one or more (in this example two) particle count locations 31, 32 (also called “benchmark locations”), which may be a professional particle monitoring station, e.g. a pollen count station.

The device 10 further comprises a particle concentration map unit 12, e.g. a data interface or a processor, for receiving or generating a particle concentration map 40 of a region 30 including the actual location 33, i.e. the location at which the device 10 currently is located. Said particle concentration map 40 includes relative particle concentration information 41 indicating, per sub-region of a number of sub-regions 34 of the region 30, the particle concentration at the sub-region relative to a particle count at one or more particle count locations 31, 32. In the example shown in FIG. 3, the particle concentration map 40 includes relative particle concentration information 41 indicating the deviation of the particle concentration at the respective sub-region 41 relative to a particle count at the particle count location 31, expressed as a percentage. For instance, the value “+15” of the relative particle concentration information 41′, assigned to sub-region 34′, means that the particle concentration at the sub-region 34′ is 15 percent higher than the particle count at the particle count location 31.

The device 10 further comprises a sub-region determination unit 13 for determining the sub-region 41, in which the actual location 33 is located, which—in this example—is the sub-region 34′. For this purpose, in an embodiment GPS data 22 acquired or received by the device 10 and/or a user input 23 indicating the actual location may be used to determine the actual location and to determine the sub-region 34′.

The device 10 further comprises a particle concentration determination unit 14 for determining the local particle concentration 24 at the actual location 33 based on the relative particle concentration information 41′ of the determined sub-region 34′ and the received particle count information 21 (acquired recently at the particle count location 31). In this example, the received particle count information 21 is multiplied by 1.15 to obtain the local particle concentration 24.

In another embodiment the same steps as explained above for the device 10 (i.e. an embodiment of a user device 2 as shown in FIG. 7) may be carried out by the remote device 3, e.g. a server in the cloud, i.e. the device 10 may also represent a remote device 3. For this purpose the user device 20 transmits its actual location to the remote device 3, which then carries out the steps to estimate the particle concentration at the actual location of the user device 2 and send the result back to the user device 2.

In still another embodiment the user device 2 represents the device 10 and obtains the actual particle concentration map from the remote device 3 and then estimates the particle concentration at the location of the user device 2.

FIG. 4 shows a map 50 of the region 30 filled with actual values 51 of the local particle concentration in the various sub-regions 34 based on the particle concentration map 40 shown in FIG. 3. These actual values are derived at a time, at which the level 52 at the benchmark location (i.e. at the particle count location 31) is 100. Applying the percentage values 41 from the particle concentration map 40 shown in FIG. 3 to this level 52 results in the actual values 51 of the local particle concentration. For instance, for the sub-region 34′ the actual value 115 (=100×115%) is obtained for the local particle concentration 51′.

Instead of providing percentage values as relative particle concentration information 41 in the particle concentration map 40, as shown in FIG. 3, in another embodiment of a particle concentration map actual values for each sub-region 34 are provided as relative particle concentration information. These actual values indicate how much has to be added to or subtracted from the received particle count information in absolute terms (e.g. an actual value of +20 means that an absolute value of 20 has to be added to the absolute value of the received particle count information acquired at a particle count location.

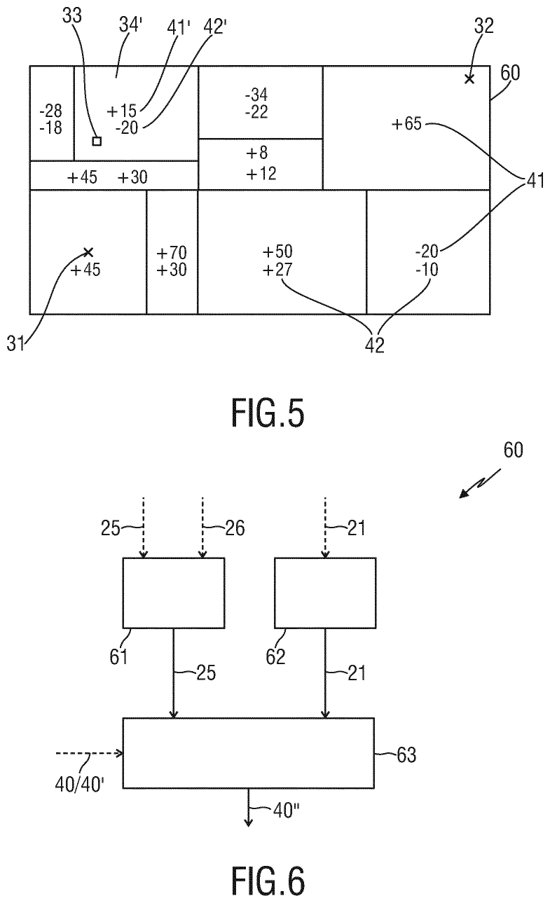

In the above explained embodiment, a recent particle count at a single particle count locations 31 and a particle concentration map 40 are used for determining the local particle concentration 24 at the actual location 33. In other embodiments, two or more recent particle counts at two or more particle count locations 31, 32 may be used. In this case a particle concentration map 40′ may be used as shown in FIG. 5, which includes two or more relative particle concentration information values 41, 42 per sub-region 34, one per particle count location indicating the particle concentration at the sub-region relative to a particle count at the respective particle count location. In other words, the relative particle concentration information values 41 relate to the particle count location 31 and the relative particle concentration information values 42 relate to the particle count location 32. For instance, for the sub-region 34′ the relative particle concentration information value 41′ of +15 means that the received particle count at the particle count location 31 has to be multiplied by 1.15 (i.e. +15%) and the relative particle concentration information value 41′ of −24 means that the received particle count at the particle count location 32 has to be multiplied by 0.80 (i.e. −20%).

Alternatively, two or more separate particle concentration maps may be used, one per particle count location.

The local particle concentration 24 at the actual location 33 may in such an embodiment be determined by extrapolating, individually per particle count location 31, 32, the particle count indicated by the received particle count information 21 based on the respective relative particle concentration information 41, 42 of the determined sub-region 34 with respect to the respective particle count location 31, 32 and by combining the extrapolated particle counts, in particular by averaging or weighted averaging the extrapolated particle counts. For instance, if the received particle count at the particle count location 31 is 100 (as shown in FIG. 5) and the received particle count at the particle count location 32 is 60, the local particle concentration at the actual location 33 will be 100×1.15 −60×0.80 =67.

In another embodiment only particle count information at the particle count location 31 closest to the actual location 33 is used for determining the local particle concentration 24 at the actual location 33.

The particle concentration map is preferably not fixed, but includes relative particle concentration information for different times, in particular different weeks and/or days and/or hours, over the year. This is particularly useful if the pollen concentration shall be determined since the distribution of pollen varies to a large extent over time, e.g. over the year. Further, the values recorded in the particle concentration map may be updated over time, e.g. continuously, based on information collected by users, mobile particle count equipment, etc.

The particle concentration map may further include relative particle concentration information for different kinds of particles, e.g. different kinds of pollen.

As an additional input, the proposed device and method may receive particle calendar information, e.g. from a pollen calendar, indicating typical particle concentrations at various regions per time. This additional input can then be taken into account when determining the local particle concentration at the actual location to further improve the accuracy of the prediction. Data from historical pollen maps may be used to improve the accuracy or reliability of the estimate, e.g. by calculating a weighted average of pollen counts (for a specific pollen type) per region. For instance if a user device (which will usually be less reliable than a monitoring station) sends a value to the system, this value could be used together with historical values for this location at same day of year to build an average.

The present invention may also be used to leverage a pollen calendar. Once a particle concentration map is available, the benchmark location can be used to get actual numbers/concentrations and a pollen calendar to indicate the pollen type for each location at a specific time/season. This and the symptom-based method can be used alternatively or together (the symptom-based approach would result in a better spatial resolution since pollen calendars are obtained from “benchmark locations”).

FIG. 6 shows a schematic diagram of a device 60 for generating or refining a particle concentration map of a region indicating the concentration of pollen and/or microorganisms, which represents an embodiment of the remote device 3 in this example. The device 60 comprises a local particle count information unit 61 for measuring and/or receiving (e.g. from a stationary monitoring unit or from a website) local particle count information 25 indicating the particle count at the actual location. The device 60 further comprises a particle count information input 62, e.g. a wireless data interface, for receiving particle count information 21 indicating a recent particle count at one or more particle count locations 31, 32. Further, the device 60 comprises a particle concentration map processing unit 63 for generating or refining a particle concentration map 40 (or 40′) of a region 30 including the actual location 33. The particle concentration map 40 (or 40′) is hereby generated or refined based on the local particle count information 25 and the received particle count information 21, resulting in a refined particle concentration map 40″.

In addition, in an embodiment, further local particle count information 26 indicating the particle count at one or more further locations may be measured and/or received. The further local particle count information includes location information indicating the location of measurement of the respective local particle count information 26 and may additionally be used in the generation and/or refinement of the particle concentration map 40 (or 40′). The further local particle count information may e.g. be acquired by wearable sensors worn by users or fixed sensors in e.g. apartments, balconies or gardens distributed throughout a region, etc.

According to this aspect of the present invention it is required to have a measurement of the concentration at the actual location at some point it time in order to generate the value for the actual location in the particular concentration map. Measurements can be performed by the community of users leading to a continuous improvement of the map (as more data come in for each location over time, the values can be statistically treated, in the simplest case averaging an increasing number of readings for each location).

According to another embodiment the particle concentration map may be generated as an initial mapping of a whole region, e.g. a whole city. A monitoring vehicle (similar to a google map car) drives through the region, e.g. a city to collect local particle count information. To obtain pollen measurements it would need to remain at each sub-region for a certain time, e.g. 60 minutes, to determine the local particle count. The obtained local particle count information is then compared to the benchmark location count to get a relative value, and the relative value is stored together with the coordinates of the corresponding location. Once this is done for the entire region (city), the obtained data base is the basis for the particle concentration map. Now, at any time point in the future, a user can send a request to the server together with his location data (without the need to send particle count information), the server determines the current concentration at the user's location based on the current benchmark location concentration and the stored relative particle concentration map, and then sends the result back to the user.

Each of the devices 10 and 60 may be implemented in hard- and/or software, e.g. as an application program running on an electronic user device, such as a smartphone, tablet, laptop, smart watch, etc. Both devices may also be combined into a single device, e.g. a single application program may be configured to carry out different methods implemented in the devices 10 and 60. In other embodiments the devices 10 and 60 are separate devices, e.g. the device 10 may be smartphone carried around by the user and the device 60 may be a computer or server, e.g. in the cloud, used as a central evaluation means.

The devices and methods can be configured in such a way that the time point where sampling of a local particle count is initiated is captured, optionally together with the time point where sampling is stopped. This can e.g. be implemented by manual input into the device by the user or, in other embodiments, automatically, e.g. the device or a local measurement station can have means which enable it to detect when a new sampling cycle begins and stops. Corresponding wireless communication means may be provided for communication between the local measurement station and the device, if needed. The sampling start event can activate location tracking (e.g. via GPS).

In an embodiment, as soon as the data (e.g. quantity and type of pollen/microorganisms) are digitalized by the device, this information is stored by the device together with the corresponding location data. Once enough data have been collected and (optionally) e.g. uploaded to a central database or server, a concentration map can be created (or refined) combining the data of multiple users. This not only further increases the spatial resolution, but can also be used to pinpoint areas of higher/lower particles than e.g. city level measurements based on the overlap between different users. An additional advantage of this approach is that for every location with overlap, data from several users can be used to increase the accuracy of the measurements (e.g. using mean values and standard deviation).

Since now a measure for uncertainty may be available, this information can be sent back to the individual users and applied to the measurements of the single user. The result is that instead of having a single value for the concentration obtained in this way, statistical information can be added to this single point measurement and a concentration range or confidence interval can be presented. This information can be also fed back to an exposure assessment performed by the device of the user to reflect the level of uncertainty.

The user interface in the application could then use this data to show a particle concentration map with more accurate concentration data and much higher spatial resolution. Such a detailed map would be very valuable for pollen, since it could for instance be used by asthma or pollen application programs (“apps”) to help people to minimize exposure to allergens by telling them which areas, namely the hot spot areas with high concentrations as determined by the device and method) to avoid, e.g. when going on a walk, doing sport etc.

Another aspect of the present invention includes concentration profiles (or maps) for a region, city etc. This may be done by comparing the values as determined by the device and method with published values from public monitoring stations. Hence, a delta may be determined for each location and expressed in percent of the published data. This may be subsequently used during days with less crowd-sourcing data to maintain a similar degree of spatial resolution. Such an approach makes particularly sense in the case of pollen, since certain areas are very likely to be always characterized by higher pollen concentrations than the ones from central monitoring stations (e.g. parks with a lot of flowers, grasses or threes).

Furthermore, if users input their symptoms (e.g. select from three smiley face options), then symptoms can be linked to particular types of particles and, based on the above, to location.

The presented approach is particularly applicable for all particles, in particular pollutants, which have fixed source locations and where the source strength is somewhat defined. For instance, pollen originate from relatively fixed sources, e.g. parks, and the differences in source strengths are also constant since determined e.g. by the number of trees at each location, size of the grass-covered area in each park etc.

The present invention may e.g. be applied in pollen pre-concentrators in air purifiers, smart-phone based pollen sensors, and smart-phone based sensors for microbiological purity of water/liquids, air and surfaces. The present invention may preferably be implemented as digital solution such as an application program. Some embodiments leverage crowdsourcing-based pollen and/or microorganism measurements (incl. pollen type) and the corresponding location data acquired during sampling to realize a number of additional benefits such as further increase in spatial resolution, increasing accuracy and providing indicators for precision. Once available, such data can be leveraged to improve existing and future application programs s for asthma and allergy management, e.g. by providing user's valuable information for trigger avoidance.

One of the ideas of the present invention is that the particle concentration map is created and constantly updates by the community of users, i.e. the particle concentration map is continuously improved by comparing each new data point coming from a specific user with the benchmark location and creating/updating the relative value at an actual location, hence the particle concentration map, based on this comparison. A corresponding data base comprising relative values per location would be preferably stored on a server, where it is updated every time a new reading comes in from a user. In this case the creating (and/or updating) the particle concentration map and the determining of the concentration at the actual position may happen at the same time.

In real life scenarios this will be difficult in most cases. Whenever the user does not remain for extended periods of time, e.g. one hour, at a fixed location, but walks/travels around and constantly changes his location, this will be difficult because current pollen sensors (even professional ones) do not provide real-time data. Hence, if a user is on the move, he could access the server at each new location and instantly receive the updated concentration at his location, without actually contributing in the map generation. The present invention thus allows determining the pollen concentration at any location at any time, which is one of the key benefits compared to known solutions.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A method of estimating a local particle concentration indicating the local concentration of pollen and/or microorganisms at an actual location, said method comprising:
receiving particle count information indicating a recent particle count at one or more particle count locations,
receiving or generating a particle concentration map of a region including the actual location, said particle concentration map including relative particle concentration information indicating, per sub-region of a number of sub-regions of the region, the particle concentration at the sub-region relative to a particle count at one or more particle count locations,
determining the sub-region, in which the actual location is located, and
determining the local particle concentration at the actual location based on the relative particle concentration information of the determined sub-region and the received particle count information.

2. The method as claimed in claim 1,
wherein the local particle concentration at the actual location is determined based on the relative particle concentration information of the determined sub-region and the received particle count information at the particle count location closest to the actual location.

3. The method as claimed in claim 2,
wherein the local particle concentration at the actual location is determined by extrapolating the particle count indicated by the received particle count information at the particle count location closest to the actual location based on the relative particle concentration information of the determined sub-region.

4. The method as claimed in claim 1,
wherein the local particle concentration at the actual location is determined based on the relative particle concentration information of the determined sub-region and the received particle count information at two or more particle count locations closest to the actual location.

5. The method as claimed in claim 4,
wherein the local particle concentration at the actual location is determined by extrapolating, individually per particle count location, the particle count indicated by the received particle count information based on the respective relative particle concentration information of the determined sub-region with respect to the respective particle count location and by averaging the extrapolated particle counts, in particular by averaging or weighted averaging the extrapolated particle counts.

6. The method as claimed in claim 1,
wherein the particle concentration map includes relative particle concentration information indicating, per sub-region of a number of sub-regions of the region, a deviation of the particle concentration at the sub-region from the particle count at one or more particle count locations in absolute or relative terms.

7. The method as claimed in claim 1,
wherein the particle concentration map includes relative particle concentration information for different times, in particular different weeks and/or days and/or hours, over the year.

8. The method as claimed in claim 1,
wherein the particle concentration map includes relative particle concentration information for different kinds of particles.

9. The method as claimed in claim 1,
further comprising the step of receiving particle calendar information indicating typical particle concentrations at various regions per time,
wherein the local particle concentration at the actual location is determined based on the relative particle concentration information of the determined sub-region, the received particle count information and the received particle calendar information at the determined sub-region.

10. The method as claimed in claim 1,
further comprising the step of measuring or receiving local particle count information indicating the particle count at an actual location,
wherein the particle concentration map is generated or refined based on the local particle count information and the received particle count information indicating a recent particle count at one or more particle count locations.

11. A method of generating or refining a particle concentration map of a region indicating the concentration of pollen and/or microorganisms, said method comprising:
measuring and/or receiving local particle count information indicating the particle count at the actual location,
receiving particle count information indicating a recent particle count at one or more particle count locations, and
generating or refining a particle concentration map of a region including the actual location, said particle concentration map including relative particle concentration information indicating, per sub-region of a number of sub-regions of the region, the particle concentration at the sub-region relative to a particle count at one or more particle count locations, wherein the particle concentration map is generated or refined based on the local particle count information and the received particle count information.

12. The method as claimed in claim 11,
wherein further local particle count information indicating the particle count at one or more further locations is measured and/or received, wherein the further local particle count information includes location information indicating the location of measurement of the respective local particle count information, and
wherein the further local particle count information is used in the generation and/or refinement of the particle concentration map.

13. A device for estimating a local particle concentration indicating the local concentration of pollen and/or microorganisms at an actual location, said device comprising:
a particle count information input for receiving particle count information indicating a recent particle count at one or more particle count locations,
a particle concentration map unit for receiving or generating a particle concentration map of a region including the actual location, said particle concentration map including relative particle concentration information indicating, per sub-region of a number of sub-regions of the region, the particle concentration at the sub-region relative to a particle count at one or more particle count locations,
a sub-region determination unit for determining the sub-region, in which the actual location is located, and
a particle concentration determination unit for determining the local particle concentration at the actual location based on the relative particle concentration information of the determined sub-region and the received particle count information.

14. A device for generating or refining a particle concentration map of a region indicating the concentration of pollen and/or microorganisms, said device comprising:
a local particle count information unit for measuring and/or receiving local particle count information indicating the particle count at the actual location,
a particle count information input for receiving particle count information indicating a recent particle count at one or more particle count locations, and
a particle concentration map processing unit for generating or refining a particle concentration map of a region including the actual location, said particle concentration map including relative particle concentration information indicating, per sub-region of a number of sub-regions of the region, the particle concentration at the sub-region relative to a particle count at one or more particle count locations, wherein the particle concentration map is generated or refined based on the local particle count information and the received particle count information.

15. A computer program comprising program code means for causing a computer to carry out the steps of the method as claimed in claim 1 when said computer program is carried out on the computer.
